(19) United States
(12) Patent Application Publication
(10) Pub. No.: US 2026/0023019 A1
(43) Pub. Date: Jan. 22, 2026

(54) DETECTION SYSTEM FOR AMINES IN FOOD PRODUCTS

(71) Applicant: MICAMO LAB S.R.L., Genova (IT)

(72) Inventors: Micaela TISO, Genova (IT); Massimo POLLIO, Milano (IT); Christian TAVELLA, Ronco Scrivia (IT); Alice POGGI, Albisola Superiore (IT); Paola LOVA, Genova (IT); Davide COMORETTO, Milano (IT); Andrea ESCHER, Genova (IT); Elena GRASSELLI, Genova (IT)

(21) Appl. No.: 18/997,314

(22) PCT Filed: Jul. 19, 2023
(86) PCT No.: PCT/IB2023/057363

§ 371 Date: Jan. 21, 2025

(30) Foreign Application Priority Data
Jul. 21, 2022 (IT) 102022000015411

(51) Int. Cl. G01N 21/64; G01N 33/02

(52) U.S. Cl. CPC G01N 21/6428; G01N 33/02; G01N 2021/6439

(57) ABSTRACT

The present invention relates to novel detection systems for amine compounds for monitoring the storage condition of food products.

The present invention relates to novel detection systems for amine compounds, in particular biogenic amines (ABs), for monitoring the storage condition of food products.

### State of the Art

Some smart packaging technologies or smart packaging are capable of providing indications of the storage conditions and/or quality of the food contained in the package. Unfortunately, although these technologies can be critical for assessing the characteristics of food products, they are strongly opposed by large-scale retail trade, who fear that consumers will not purchase products with negligible alteration changes, resulting in large amounts of food waste.

ABs are important nitrogen compounds formed mainly by the decarboxylation of amino acids or the amination and transamination of aldehydes and ketones. In foods and beverages, biogenic amines are mainly formed by the decarboxylation of amino acids, mainly by microorganisms. Therefore, the total amount and variety of amines in food packages strongly depend on the type of food, on the microorganisms present, and on the preservation state of the food (Yesim Özogul et al., Biogenic Amines in Food: Analysis, Occurrence and Toxicity, 2019, pp. 1-17). ABs can be present in many products such as meat, vegetables, fish products, dairy products, wine, and fermented products. The latter, for example, may contain varying amounts of different amines even when subjected to heat treatment. In addition, some products such as mackerel and non-mackerel fish (e.g., sardines, salmon, swordfish, anchovies, herring), cheese, meat products, and beverages may contain high levels of biologically active amines, i.e., be harmful to consumers, and are therefore considered risky foods (Ingars Reinholds et al., Foods 2020, 9(1), 93). For example, numerous biogenic amines can be formed in the muscle tissue of fish. One of these is histamine, which is formed by the decarboxylation of the amino acid histidine. This occurs through the action of enzymes such as histidine decarboxylase, which is produced or released by some bacteria such as Escherichia coli, Vibrio, Proteus, Klebsiella, Clostridium, Salmonella and Shigella (Masashi Kanki et al., Appl Environ Microbiol. 2007 March; 73(5): 1467-1473). In this regard, Regulation (EC) No. 2073/2005 identifies red meat fish species such as tuna, mackerel, sardines, herring and dolphinfish as those most at risk for the presence of histidine. The presence of histamine can also be detected in fish families not mentioned in the regulation (e.g., white-fleshed fish such as amberjack) if improperly stored over a long period of time. In addition to histamine, protein degradation also produces other biogenic amines such as cadaverine and putrescine, which have a synergistic or potentiating effect of histamine and impart an unpleasant smell to the altered product. However, the toxic effect is maintained only by histamine. Note that fresh fish does not contain histamine or concentrations less than 10 mg/kg. Therefore, the increase in concentration is due to the following factors:
1. use of raw materials that are no longer fresh,
2. time/temperature abuse,
3. use of enzymatic maturation processes (proteolysis) in the production of processed foods (e.g., salted anchovies) that lead to the formation of free histidine in the product, further promoting histamine production.

In particular, the influence of temperature on the formation of AB in fish is crucial. In fact, the formation of AB occurs at both moderate and high temperatures, with histamine formation being highest at temperatures of 37.8° C. Conversely, storage of products at low temperatures is unfavorable for the formation of amines; if fish products are stored at 0° C., they contains lower amounts of histamine for up to 18 days, if they are stored at 10° C., they have concentrations of up to 1 g/Kg already after only 5 days. According to EC Reg. 2073/05, 100 mg/kg of histamine in histidine-rich fishery products, such as the mackerel family, represents a potentially toxic level. Also in cheese, the availability of amino acids together with pH and salinity is favorable for the formation of AB. In this case, however, the values found are lower than those considered toxic, but can greatly alter the organoleptic characteristics of the food. Therefore, the problem of the presence of amines in dairy products should not be underestimated.

Cadaverine can be used to monitor alteration in white and red meat (Vinci G., Control. 2002;13:519-524), whereas tyramine can only be used for red meat. In chicken meat, amines increase earlier and faster than in beef (Alessandroni L. et al., Food Chem. 2022;371:131134), and therefore storage of this meat is more critical. The increase in total AB content is also temperature dependent. In fact, storage of meat at a temperature below 4° C. can reduce the formation of AB (Maria Schirone et al., Foods 2022 March; 11(6): 788). The increase in putrescine, cadaverine, and histamine levels in pork also appears to correlate significantly with the concentration of total volatile basic nitrogen (TVB-N). In fresh beef vacuum-packed and stored at 1° C. for 120 days, the formation of amines is observed at significant levels from the 20th day of storage. In salami and soppressata from southern Italy, the most abundant amine is tyramine (up to 500 mg/Kg), followed by putrescine and cadaverine, 2-phenylethylamine was found in very low amounts and histamine only in some soppressata samples (50 mg/Kg).

Considering that AB can be ingested in the diet from a variety of sources, the sum of amines ingested by the consumer can easily exceed safe levels. Consequently, when assessing the risk associated with amine intake, it is not individual amines but their sum that should be considered (Agata Durak-Dados et al., J Vet Res. 2020 June; 64(2): 281-288).

Thus, it becomes clear that there is a need to provide new detection systems for amine compounds to monitor the preservation status of foods. In this context, the evolution of amines in foods of animal and plant origin from the earliest degradation processes can be used to indicate poor preservation or handling of a food even before it is spoiled, so that it can be consumed in a short time, minimizing food waste and protecting consumer health. In this context, monitoring the total amines produced by degradation processes is also important.

Several systems for detecting the state of food storage are described in the literature. For example, patent application CA 2621754 A1 describes CO2 sensors based on polar polymer matrices that contain a number of indicators, including a pH-sensitive dye and a lipophilic metal ion-cation complex. These receptors form a soluble ion pair in polar polymer matrices, such as ethylcellulose, methylcellulose, and aminocellulose. In this case, the materials are processed from solution with toxic solvents such as methanol.

A review by Danchuck et al. Analytical and Bioanalytical Chemistry (2020);412:4023-4036 discusses research by Severin et al. Chem. Commun. (2011);47:9639-9641, which focuses on the use of coumarin derivatives prepared by formylation of 7-(N,N-dimethylamino)-4-hydroxycoumarin. The modification of hydroxycoumarin in this case allows the molecule to react with amines in buffered aqueous solutions to form enamines. The reaction to some amines shows a color change in the system, which is clearly visible to the naked eye, as the molecules absorb visible light up to 500 nm. Formylated hydroxycoumarin also shows a different color change for different amines and vapor phase ammonia when incorporated into polymethyl methacrylate (PMMA) deposited from a chloroform solution. PMMA is a polymer used for decorative purposes because of its high optical transparency, but is treated at least once with formylated hydroxycoumarin in chloroform. However, the process and materials used are not compatible with use in food packaging because the solvent (chloroform) needed to make the film is highly toxic, teratogenic and suspected carcinogenic. The PMMA used also does not appear to be suitable for use in food, nor does the new formylated compound, for which no information is available on the toxicity properties and possible use as a contact material.

Hongqi Li et al. (“Coumarin-Derived Fluorescent Chemosensors,” Advances in Chemical Sensors (2012)) reviewed a paper by Richard J. Ansell et al. Org. Biomol. Chem. (2009); 7(6): 1211-1220 on the polymerization of 6-styrylcoumarin-4-carboxylic acid (SCC) and 6-vinylcoumarin-4-carboxylic acid (VCC) into “molecularly imprinted polymers” (MIPs) that are precisely polymerized around a molecule of (+)-ephedrine. This polymerization process of SCC and VCC derivatives allows the creation of specific porosities bearing the shape of the ephedrine molecule. This property means that, due to the nature of MIPs, the system can only interact with the molecule used for the synthesis, namely the (+)-ephedrine enantiomer. In fact, the systems do not even react with the (−)-ephedrine enantiomer, which is structurally identical to the enantiomer used, except for the different spatial arrangement of some bonds, let alone with other amines. Again, the reaction of the system is clearly visible colorimetrically and to the naked eye, as the emission spectrum of the polymer shows peaks in the green spectral region. In principle, the process can be applied to individual biogenic amines, but the polymerization of 6-styrylcoumarin-4-carboxylic acid (SCC) and 6-vinylcoumarin-4-carboxylic acid and molecular imprinting are rather complex processes that are not compatible with food packaging because they involve the use of several toxic and potentially carcinogenic compounds and solvents. In fact, there are no studies on the toxicity of the new coumarin-based polymers. Moreover, the extreme selectivity of MIPs does not allow their use for the detection of total biogenic amines, which is essential to enable the systems to detect the storage status of different protein matrices such as meat, fish and cheese.

From the analysis of the existing literature it is therefore clear that the currently proposed systems are based on the use of chemical compounds that prevent their use in packaging due to problems related to potential toxicity. Furthermore, all systems are developed on colorimetric detection, visible to the naked eye. This approach has already failed as it has been shown to lead to increased food waste.

### SUMMARY OF THE INVENTION

A purpose of the present invention is to provide a product capable of solving the above-mentioned problem of early detection of food spoilage in an innovative and original way, through the detection of total amines produced by systems compatible with food packaging, i.e., non-toxic and biocompatible, and undetectable to the human eye without the use of appropriate instruments. For this purpose, a sensor for the detection of amines is introduced into the packaging, aimed at detecting degradation processes and based on an optical reaction that is not visible to the naked eye, i.e., non-colorimetric and preferably fluorimetric.

Another purpose of the present invention is to provide a system comprising a detection sensor consisting of a polymer matrix, a chemical receptor, and a simple readout device that can be monitored only by insiders.

The sensor is part of the package itself and has a negligible impact on its cost. In addition, the sensor is inherently capable of tracking the end-of-life of the packaging. Thus, the status of the individual package can be comprehensively monitored throughout the supply chain.

Both the polymer matrix and the receptor are “food grade,” meaning they can be used as food contact materials.

No toxic solvents or molecules are used in the manufacture of the system, only molecules and solvents that comply with regulations and specifications approved for use in the food industry. Thus, the components of the system are safe and suitable to come into contact with food without causing risks to human health or altering the safety or quality of food.

The selected polymers meet requirements such as the absence of toxic substances or contaminants, chemical stability and food compatibility. They follow regulations and guidelines, such as those of the Italian National Unification Body (UNI) or the European Food Safety Authority (EFSA), to ensure safety and compliance with food standards.

The equipment used to detect the sensor condition consists of a monochromatic light source whose emission is compatible with the absorption spectrum of the chemical receptor, only after interaction with the degradation products of the food. The selected wavelength must not be absorbed by other materials, including the polymer matrix, the receptor that has not interacted with the food degradation products, the food itself, and those resulting from the degradation of the food.

The principal purposes described above are achieved with a sensor according to claim 1, with its use for detecting total amines in a food according to claim 10, with a method of making the sensor according to claims 11 and 12, with a food container containing the sensor according to claim 13, and with a detection system according to claim 14.

### DESCRIPTION OF THE FIGURES

FIG. 1: Absorption spectrum of maltol (black, solid line) and coumarin (gray, dashed line) in PLA (left, panel a) and CA (right, panel b).

FIG. 2: Coumarin absorption spectrum before (black solid line) and after (gray dashed line) interaction with an amine.

FIG. 3: Photoluminescence spectrum of PLA (panel a) and PS (panels b, c and d) films containing coumarin (a, b), maltol (c) and kojic acid (d) before (black solid line) and after (gray dashed line) interaction with an amine in the vapor phase.

FIG. 4: Infrared spectrum of a PLA film before and after interaction with an amine.

### DEFINITIONS

Unless otherwise defined, all terms of art, notations and other scientific terminology used herein are intended to have the meanings commonly understood by those of skill in the art to which this disclosure pertains. In some cases, terms with commonly understood meanings are defined herein for clarity and/or for ready reference; thus, the inclusion of such definitions herein should not be construed to represent a substantial difference over what is generally understood in the art.

The terms “approximately” and “about” herein refers to the range of the experimental error, which may occur in a measurement.

The terms “comprising”, “having”, “including” and “containing” are to be construed open-ended terms (i.e. meaning “including, but not limited to”) and are to be considered as providing support also for terms as “consist essentially of”, “consisting essentially of”, “consist of” or “consisting of”.

The terms “consist essentially of”, “consisting essentially of” are to be construed as semi-closed terms, meaning that no other ingredients which materially affects the basic and novel characteristics of the invention are included (optional excipients may thus included).

The terms “consists of”, “consisting of” are to be construed as closed terms.

The term “food grade” means that it can be used as a food contact material according to national and international regulations.

### DETAILED DESCRIPTION OF THE INVENTION

It is an object of the present invention to provide a non-colorimetric optical sensor for detecting the presence of total biogenic amines, consisting of a food grade polymer matrix having dispersed therein a molecule containing one or more pyrones (receptor) which are not chemically bound to the matrix.

In a preferred embodiment, the sensor according to the invention is fluorimetric and not visible to the naked eye, i.e. not colorimetric.

Advantageously, the molecule containing one or more pyrones is capable of changing its optical properties (absorption spectrum and fluorescence) upon selective interaction with amine groups of amines formed during food degradation. Thus, the sensor detects all developed amines and is non-specific.

In particular, the receptors used are associated with light absorption phenomena below 400 nm, depending on the structure of the molecule (receptor) used.

This property makes it possible to detect the presence of biogenic amines with simple instruments by a change in the absorption and/or fluorescence spectrum of the receptor, which is invisible to the naked eye and therefore to the consumer.

The detection of amine compounds is essential for monitoring the condition of food (meat, fish, cheese and processed products in general, including alcoholic and non-alcoholic beverages).

In a preferred embodiment, the polymeric matrix is selected for its food compatibility, chemical resistance, thermal stability, and other properties that make it suitable for use in the food environment. Preferably, the polymeric material is selected from polystyrene (PS), polypropylene (PP), polyethylene (PE), polyethylene terephthalate (PET), polylactic acid (PLA), or mixtures thereof. Thus, the polymers used are exclusively “food grade” and do not exhibit any particular polarity properties that could cause interactions between the matrix and foods that are predominantly water-based.

In another preferred embodiment, the sensor according to the invention is able to detect the presence of concentrations of total amines between 500 micrograms per kilogram and 10 grams per kilogram in the packaging atmosphere or under ambient conditions, in order to detect the onset of degradation processes as soon as they are triggered, in such a way as to facilitate the implementation of avoidance/disposal/expiry date reduction measures by the food business operator, in order to protect consumer health while minimizing food waste.

In another preferred embodiment, the polymer matrix is in the form of films (preferably 100 nm-500 μm thick), fibers, films, sheets, foams, pellets or powders compatible with the specific application.

Preferably, the receptor containing one or more pyrones that can be used in the present invention is selected from coumarin, maltol, ethylmaltol, chromone or kojic acid, and flavonoids, which are generally extracted from natural material and not chemically treated. Pyrones are molecules consisting of a six-term hetero-ring containing an oxygen atom and a ketone functional group.

Examples of commercially available pyrone-containing molecules that can be used in the present invention include coumarin (1-benzopyran-2-one), maltol (3-hydroxy-2-methyl-4H-pyran-4-one) and its derivatives such as ethyl maltol (2-ethyl-3-hydroxy-pyran-4-one), chromone (1,4-benzopyron), kojic acid (5-hydroxy-2-(hydroxymethyl)-4H-pyran-4-one) and/or flavonoids.

The receptor is dispersed in the polymer matrix and is not chemically bound so as not to alter its nature and thus affect its use in food packaging in accordance with current regulations.

The molecules containing pyrones are non-toxic at the concentrations used (1-20% w/w). The sensor has variable and suitable sizes to control the total amount of receptor present.

Another object of the present invention is represented by the use of the sensor to detect total amines in a food product exclusively by optical methods (absorption or fluorescence) that are not detectable by the human eye, i.e., that do not provide colorimetric responses (fluorescence emission with characteristic color or absorption in the visible spectrum).

A further object of the present invention is represented by the methods for manufacturing the sensor according to the invention comprising:
mixing a polymeric material and a molecule containing one or more pyrones in a food grade solvent, preferably according to the 2009/32/EC regulation, the Regulation (EC) n. 1935/2004 and Regulation (EC) no. 10/201 (more preferably in a solvent selected from water, ethyl acetate, ethanol, acetone, hexane, ethyl methyl ketone, diethyl ether, methyl acetate, propanol and butanol) according to the polymer matrix and the pyrone used;
depositing the resulting solution on a substrate (e.g. greaseproof paper, Teflon, glass, packaging films), preferably by techniques such as drop-casting, spin-coating and dip-coating;
evaporation of the solvent, preferably at room temperature, to form a film;
extraction of the remaining solvent by one or more washes in water and one or more vacuum treatments;
optionally, removal of the film from the substrate.

The concentration of the polymer is preferably 30-50 g/L, while the molecule containing one or more pyrones has a concentration preferably between 1 and 20% by weight, based on the weight of the polymer.

All processes are preferably carried out under ambient conditions (pressure and temperature).

Preferably, the film has a thickness between 100 nm and 500 μm.

In an embodiment, the sensor can be made from polymer melts by extrusion and subsequent deformation. In this case, the fabrication includes:
mixing the polymeric material (powder, pellet or other physical form) with the molecule(s) containing the pyrone(s) (powder, pellet or other physical form) at room temperature or at temperatures above the melting and/or glass transition temperature of the polymeric material;
homogenizing of the resulting mixture;
melting/softening of the polymer matrix containing the receptor (the molecule containing one or more pyrones);
forming films, including structured films such as those formed from fiber bundles, preferably by industrial techniques including extrusion, calendering, laminating, film casting, film blowing, blow molding, thermoforming, spinning, electrospinning, molding, rotoforming, additive manufacturing, and foaming.

Another object of the present invention is a food container containing the sensor of the invention.

The sensor may be in the form of “labels” inside the package, visible or invisible to the naked eye, or may form the package itself (films, food trays, absorbent pads) with the polymer-molecule mixture containing one or more pyrones. The sensor inherently follows the end of life of the packaging.

The sensor functions (i) in contact with the food, (ii) on the bottom of the tray in contact with the produced liquids, and (iii) even when not in contact with the food, by interaction with volatile amines in the vapor phase.

Another object of the present invention is a system for detecting total amines in a food product, comprising the sensor according to the invention, a monochromatic light source with an emission wavelength corresponding to the absorption of the receptor molecule after interaction with the amines, a non-dispersive detector (photodiode, photoresistor, photodetector, etc.) using suitable optical systems (filters and lenses) to filter the radiation incident on the sensor.

The monochromatic light source must be compatible with the absorption spectrum of the chemical receptor after interaction with food degradation products and capable of exciting its fluorescence. Preferably, it must not be absorbed by other materials, including the polymer matrix, the receptor that has not interacted with the food degradation products, the food products, and the products resulting from the degradation of the food degradation products.

The following examples are intended to further illustrate the invention, but not to limit it.

### EXAMPLES

### Materials

### Example 1

10 g of PS is mixed with 1 g of maltol and dissolved in hexane. The solution is applied to Teflon-coated paper and allowed to dry. The resulting film is peeled off the substrate and glued into a food package to form a “label” visible from the outside. When illuminated with an intense light source at 405 nm, the label emits specific radiation only when it interacts with biogenic amines from food degradation. The emission is collected and recorded by a detection system consisting of a photodiode and an optical filter.

### Example 2

100 g of PS is premixed with 10 g of coumarin and fed to an extruder. The extrusion process mollifies the polymer and homogenizes the mixture. The extrudate is filmed to form an amine-sensitive film that can serve as food packaging. Alternatively, the extrudate can be made into trays or labels with the same properties. The detection is carried out as described in Example 1.

### Methods

Reading of the sensor can be done by various methods based on fluorescence and absorption of light by the pyrone-containing molecule.

In absorption measurement, the polymer matrix loaded with the receptor is illuminated with a beam of white light (200-1100 nm), which is then sent to a non-dispersive detector that can quantify the absorption of the material. The value is then correlated with the values of the receptor before and after reaction with the previously detected amines.

In the fluorescence measurement, the polymer matrix containing the receptor is illuminated with a high-intensity monochromatic light beam to excite the fluorescence of the receptor. Fluorescence can then be measured with a nondispersive detector such as a photodiode or photoresistor coupled to an optical filter system that can only detect the presence/absence and intensity of fluorescence. The light emitted from the sample and any reflected light sources are filtered through a dichroic or colored filter system to ensure that the detector works and to avoid false readings.

### Results

UV-Vis and FT-IR spectroscopic analyzes show a clear response of the sensor of the invention to amine groups.

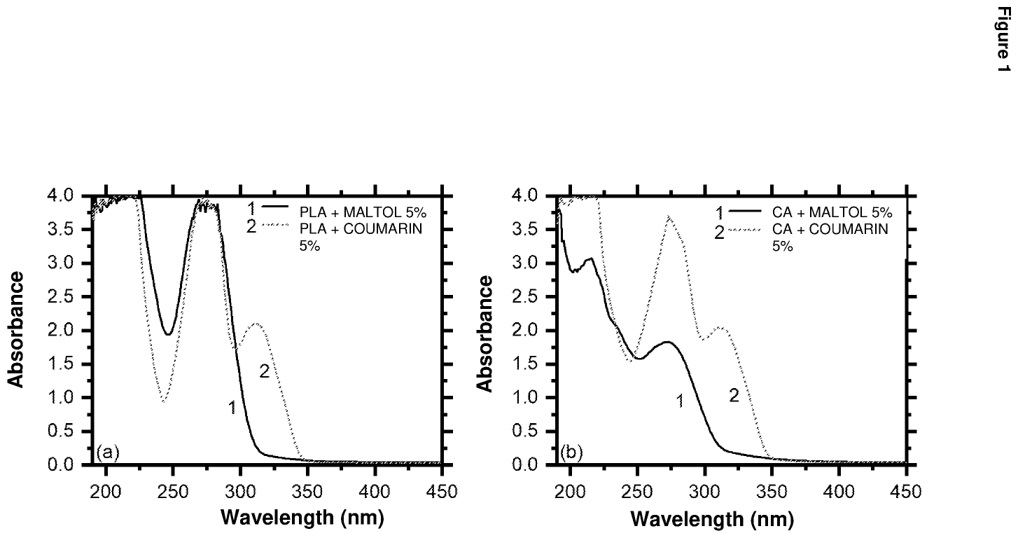

FIG. 1 shows the UV-Vis analysis for some tested materials. In the spectrum on the left PLA loaded with maltol (black, solid line) and coumarin (gray, dashed line), in the spectrum on the right cellulose acetate loaded with the same receptors. The absorption bands below 250 nm are assigned to the polymer matrices, while the receptors absorb at wavelengths between 250 and 350 nm. In fact, the films appear completely transparent.

The absorption bands imply that to stimulate the fluorescence signal of the molecule that has not reacted with the amines in the polymer matrix requires an intense light source at wavelengths below 350 nm for both molecules in both polymer matrices. Such behavior is characteristic of many pyrones covered by this invention.

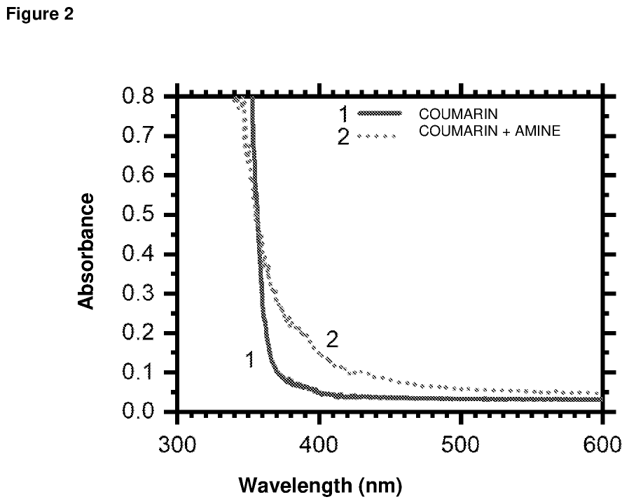

To illustrate the changes that occur during exposure to amines, FIG. 2 shows the absorption spectrum of a concentrated coumarin solution before interaction with an amine (black solid line) and after (gray dashed line). It can be seen that the coumarin molecule absorbs at longer wavelengths after reacting with the amine. This allows:
monitoring the response to the presence of amines also by intensity measurements of the absorbed radiation.
stimulating the fluorescence of the pyrone-containing molecule that has interacted with one or more ABs by the light source at wavelengths longer than 350 nm.

In this case, the fluorescence is excited only when the pyrone-containing molecule has interacted with the amine.

Thus, the presence of the fluorescence signal indicates the presence of amines and the beginning of degradation processes in the food.

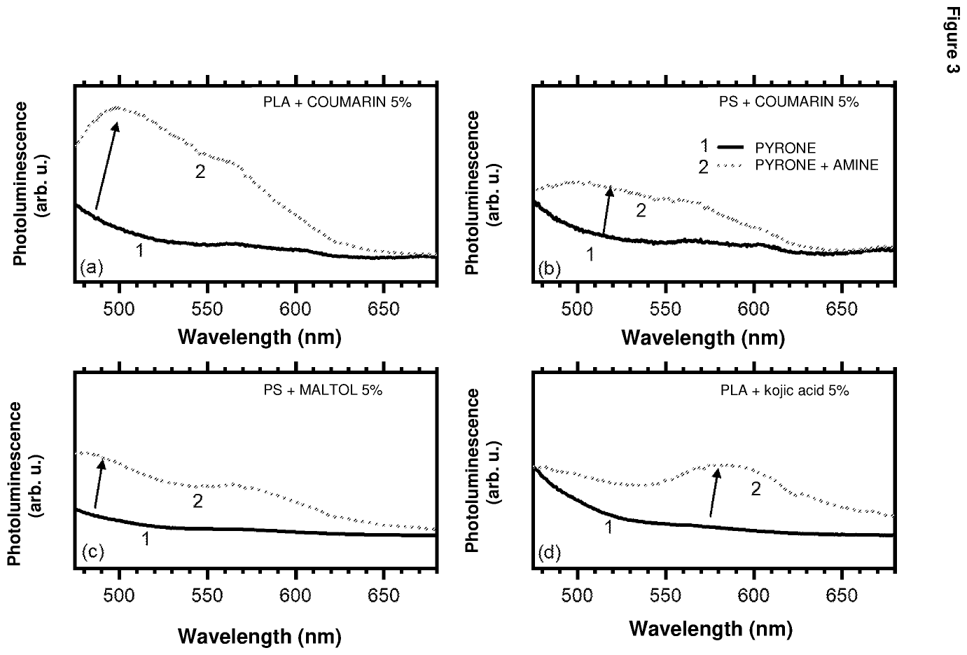

FIG. 3 shows the response of some polymeric sensors loaded with coumarin, maltol and kojic acid and exposed to vapors of an amine. It can be seen how the emission spectrum, initially peaked at wavelengths lower than the spectral range analyzed (black lines), is modified and consists of a non-colored emission but which extends over the entire visible spectrum making it impossible to interpret to the naked eye (dashed gray lines).

It is to be noted that the absorption shift of the amine allows to measure the fluorescence of the material with sources at wavelengths greater than 400 nm, only after interaction with the amine.

Fluorescence can then be stimulated with any source of wavelength less than 350 nm (before and after interaction with the amine) or with sources of higher wavelengths. In the latter case, the presence of a fluorescence signal alone, which is only possible after the interaction between the pyrone-containing molecule and the amine, indicates that the interaction has occurred.

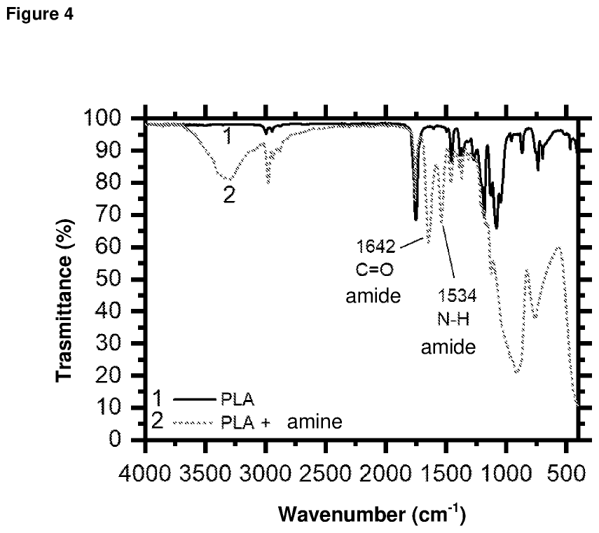

FIG. 4 shows the infrared spectrum of a PLA polymer matrix. In this single case, the polymer participates in the mechanism of amine recognition by breaking the polymer chain with mechanisms of beta-elimination or nucleophilic substitution. This allows more effective exposure of the receptor to amines. Indeed, the spectrum of PLA exposed to amine shows the presence of an enlarged band between 3000 and 3800 cm-1 associated with the presence of amine groups. In addition, the two bands at about 1640 and 1535 cm-1, characteristic of the amide group formed during polymer chain breakage, are clearly visible in the spectrum of the material collected after exposure to amine.

1. A non-colorimetric optical detection sensor for detecting total amines in a food product, consisting of a food grade polymer matrix wherein a molecule containing one or more pyrones is dispersed and not chemically bound to the polymer matrix.

2. The sensor according to claim 1, wherein it is of the fluorimetric type that is not visible to the naked eye.

3. The sensor according to claim 1, capable of emitting fluorescence only after interaction with one or more amines, which is not perceptible without suitable readout instruments.

4. The sensor according to claim 1, wherein the food is proteinaceous, preferably meat, fish, cheese, processed products or alcoholic and non-alcoholic beverages.

5. The sensor according to claim 1, wherein the food grade polymer matrix consists of a non-polar polymer material selected from polystyrene (PS), polypropylene (PP), polyethylene (PE), polyethylene terephthalate (PET) and polylactic acid (PLA) or mixtures thereof.

6. The sensor according to claim 1, wherein it detects concentrations of total amines in the foodstuff that are compatible with the establishment of degradation processes, the concentrations preferably being between 500 micrograms per kilogram and 10 g per kilogram.

7. The sensor according to claim 1, wherein the polymer matrix is in the form of films, fibers, sheets, foams, pellets or powders.

8. The sensor according to claim 1, wherein the molecule comprising one or more pyrones is selected from coumarin, maltol and its derivatives, chromone, kojic acid and/or biologically derived flavonoids and is suitable for use in food packaging according to the applicable regulations.

9. The sensor according to claim 1, wherein it is in the form of labels inside the package or forms the package itself.

10. Use of the sensor according to claim 1 for detecting total amines in a food product by optical methods that are not colorimetric and cannot be perceived by the human eye.

11. A method for making the sensor according to claim 1, comprising:
mixing a polymeric material and a molecule containing one or more pyrones in a food grade solvent, preferably selected from water, ethyl acetate, ethanol, acetone, hexane, ethyl methyl ketone, diethyl ether, methyl acetate, propanol and butanol;
depositing the resulting solution on a substrate, preferably by a technique selected from drop casting, spin coating and dip coating;
evaporation of the solvent to form a film;
extraction of the remaining solvent by one or more washes in water or one or more vacuum treatments;
optionally, removal of the films from the substrate.

12. The method for making the sensor according to claim 1, comprising:
mixing a polymeric material and a molecule containing one or more pyrones at room temperature or at temperatures above the melting and/or glass transition temperature of the polymeric material;
homogenizing the resulting mixture;
melting or softening the polymer matrix containing the molecule;
forming a film, preferably by a technique selected from extrusion, calendering, laminating, film casting, film blowing, blow molding, thermoforming, spinning, electrospinning, molding, rotoforming, additive manufacturing, and foaming.

13. A food container containing the sensor according to claim 1, wherein the sensor operates in contact with vapors, liquids, or solids containing one or more amines.

14. A system for detecting total amines in a food product comprising the sensor according to claim 1, a monochromatic light source, and a non-dispersive detector.

15. The system according to claim 14, wherein the non-dispersive detector is selected from a photodiode, a photoresistor, a photomultiplier, or a photodetector coupled to a suitable optical system that includes a filter system for radiation incident on the sensor.
